(19) United States
(12) Patent Application Publication
(10) Pub. No.: US 2024/0343849 A1
(43) Pub. Date: Oct. 17, 2024

(54) TAGGED POLYMER AND METHOD

(71) Applicant: Sterling Specialty Chemicals Holding UK Limited, Harrow, Middlesex (GB)

(72) Inventors: Mehrdad HESAMPOUR, Espoo (FI); Vesa NUUTINEN, Espoo (FI); Erkki Johannes METSÄLÄ, Espoo (FI); Salla PUUPPONEN, Espoo (FI); Viktor KUKKONEN, Espoo (FI)

(21) Appl. No.: 18/562,124

(22) PCT Filed: Jun. 1, 2022
(86) PCT No.: PCT/US2022/031811

§ 371 Date: Nov. 17, 2023

Related U.S. Application Data
(60) Provisional application No. 63/195,350, filed on Jun. 1, 2021.
(60) Provisional application No. 63/365,177, filed on May 23, 2022.

(51) Int. Cl. C08F 220/56; C09K 11/06; G01N 21/64; G01N 33/58

(52) U.S. Cl. CPC C08F 220/56; C09K 11/06; G01N 21/6428; G01N 33/582; C09K 2211/145; G01N 2021/6439

(57) ABSTRACT

Compounds, polymer compositions, systems, and methods for determining a concentration of a polymer composition in a fluid. The polymer compositions may include a copolymer that includes a tagging monomer. The copolymer may have a molecular weight of at least 1,000,000 Da (Mw). Methods for synthesizing compounds and polymer compositions.

### CROSS-REFERENCE TO RELATED APPLICATIONS

This application claims priority to U.S. Provisional Patent Application No. 63/195,350, filed Jun. 1, 2021, and U.S. Provisional Patent Application No. 63/365,177, filed May 23, 2022, which are incorporated by reference herein.

### FIELD OF THE INVENTION

This disclosure is directed to tagging agents and polymeric, detectable chemical compositions, including compositions that may be used in enhanced oil recovery and other applications, such as water treatment applications.

### BACKGROUND

In many applications, including oil sands and tailings applications, flocculants and polymers are used. Polymer enhanced oil recovery is a prominent technology that may be used to increase the production of oil. Hydrolyzed polyacryl amide (HPAM) is commonly used for polymer flooding. The injected polymer propagates from an injection well towards a production well through a reservoir. The time it takes a material to travel this path can depend on a number of factors, including one or more reservoir characteristics, such as permeability, heterogeneity, type (e.g., sand stone, carbonate, etc.), temperature, brine salinity, oil properties, etc.

Understanding a polymer's path in a reservoir can provide insight on how the polymer propagates, and how it may behave differently from models. For example, if a polymer bypasses an oil layer and enters a more permeable layer, the total polymer flooding efficiency typically is reduced. One way to understand polymer propagation and its concentration at different depths of a reservoir is to collect samples by using production logging tool (PLT) samples, but this is usually a very costly procedure that is not regularly used by operators.

The ability to measure polymer concentration during various processes can provide the possibility of avoiding under/over dosing of polymer, thereby permitting the optimization of chemical dosage.

Typically, measuring the concentration of polymers is challenging, because the desired concentration in most, if not all, streams is less than the detection limit of most, if not all, analytical equipment. Moreover, standard analytical methods, such as size exclusion chromatography (SEC), typically require extensive sample preparation and/or pretreatment before measurement.

There remains a need for improved tagged polymers, and improved methods for tagging polymers, including methods that permit concentrations to be measured accurately.

### BRIEF SUMMARY

Provided herein are compounds, which may be used as fluorescent tagging agents. The compounds, in some embodiments, can be polymerized, such as with one or more comonomers, to form polymers tagged with a fluorescent moiety. The polymers may include high molecular weight polymers, i.e., polymers having a molecular weight (Mw) of at least 1,000,000 Da. The one or more comonomers may include dispersing agents, anti-scalants, polymers, and/or other materials that may benefit from the presence of a controllable tag.

Also provided herein are methods for measuring concentrations of tagged polymers. The tagged polymers provided herein may permit the quick, easy, and/or accurate measurement of polymer concentration, including the concentration of tagged polymers having a very high molecular weight (e.g., at least 1,000,000 Da (Mw)). The compounds and polymer compositions provided herein may be suitable for a number of applications, including, but not limited to, polymer flooding, oil field anti-scalants, dispersing agents, and/or scale-controlling agents, which may optionally be used in cooling towers. The compounds provided herein and/or derivatives thereof may be used as biomarkers.

In one aspect, compounds or isomers of formula (I), formula (I′), formula (I″), and formula (II) are provided. In another aspect, polymer compositions are provided. The polymer compositions may include a copolymer. The copolymers, in some embodiments, include (i) a first monomer including (a) a compound of formula (I), formula (I′), or formula (I″), or (b) a compound or an isomer of formula (II), wherein the first monomer is a tagging monomer, and (ii) at least one second monomer including at least one polymerizable double bond or at least one polymerizable triple bond;

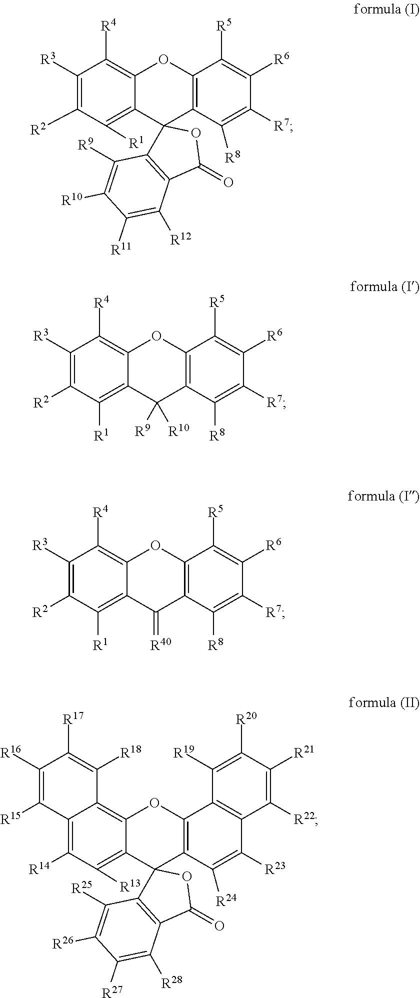

wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ are independently selected from the group consisting of hydrogen, hydroxyl, C₁-C₆ alkoxy, C₂-C₆ alkenoxy, C₂-C₆ alkynoxy, —N(R′)(R″), C₁-C₆ alkyl, C₂-C₆ alkenyl, C₂-C₆ alkynyl, and C₄-C₁₄ aryl; wherein R⁴⁰ is selected from the group consisting of C₁-C₆ alkoxy, C₂-C₆ alkenoxy, C₂-C₆ alkynoxy, —N(R′), —N(R′)(R″), C₂-C₆ alkyl, C₂-C₆ alkenyl, C₂-C₆ alkynyl, and C₄-C₁₄ aryl; wherein R′ and R″ are independently selected from the group consisting of hydrogen and C₁-C₆ alkyl, and wherein the isomers of Formula (II) include a compound of Formula (IIi), a compound of Formula (IIii), a compound of Formula (IIiii), or a combination thereof—

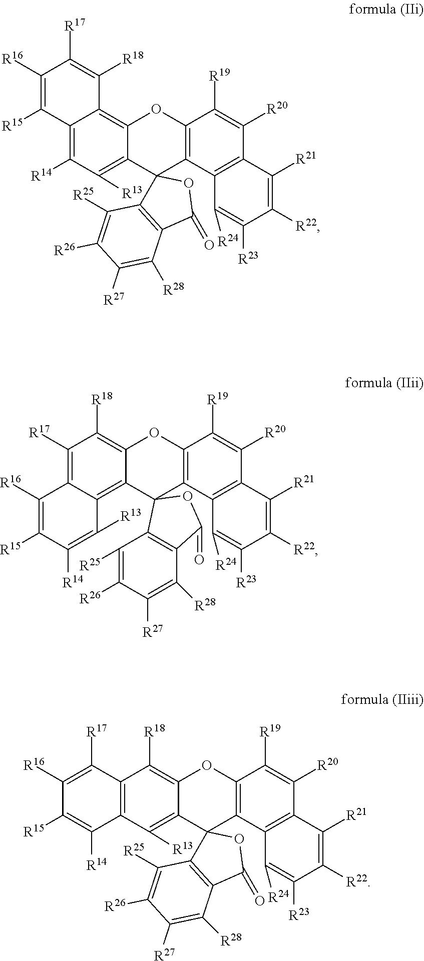

In another aspect, methods for measuring polymer concentrations are provided. In some embodiments, the methods include providing a system that includes a fluid in circulation, wherein the fluid includes a polymer composition as described herein; and measuring with an analytical technique an amount of a tagging agent monomer in the system or the fluid, wherein the measuring is performed periodically or continuously. Measuring the amount of a tagging agent monomer in the system or the fluid may permit the amount of a polymer composition that is present in the system or the fluid to be determined. The methods also may include regulating an amount of a polymer composition in a system or a fluid. For example, the methods also may include adding an additional amount of a polymer composition to the system or the fluid if the amount of the polymer composition in the system or the fluid is less than a predetermined value. The methods also may include removing an amount of a polymer composition in a system or a fluid if the amount of the polymer composition in the system or the fluid is greater than a predetermined value.

In yet another aspect, methods of synthesizing compounds and polymer compositions are provided. In some embodiments, the methods of synthesizing the compounds include a polycondensation reaction. A polycondensation reaction may include contacting an aryl alcohol (e.g., an amino-substituted aryl alcohol (e.g., an aminophenol), or a hydroxyl-substituted aryl alcohol), a condensation catalyst, and a compound according to formula (A) to form a compound (e.g., a compound of formula (I), formula (I′), or formula (I″), or a compound or isomer of formula (II)) as a condensation product:

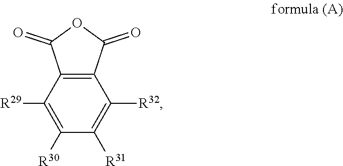

wherein R²⁹, R³⁰, R³¹, and R³² are independently selected from the group consisting of hydrogen, hydroxyl, C₁-C₆ alkoxy, C₂-C₆ alkenoxy, C₂-C₆ alkynoxy, —N(R)(R″), C₁-C₆ alkyl, C₂-C₆ alkenyl, C₂-C₆ alkynyl, and C₄-C₁₄ aryl. In some embodiments, the methods include polymerizing a compound, such as a condensation product. The methods of polymerization may include contacting a compound, including a condensation product of the methods herein, with at least one second monomer to form a copolymer; wherein the at least one second monomer includes a polymerizable double bond and/or a polymerizable triple bond.

Additional aspects will be set forth in part in the description which follows, and in part will be obvious from the description, or may be learned by practice of the aspects described herein. The advantages described herein may be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1 depicts a plot of data showing an effect of tag dosing on serum viscosity for embodiments of tagged polymers.

FIG. 2A depicts a plot of intensity v. active concentration for an embodiment of a tagged polymer.

FIG. 2B depicts a plot of intensity v. active concentration for an embodiment of a tagged polymer.

FIG. 2C depicts a plot of differential pressure v. time for an embodiment of a tagged polymer and an untagged polymer.

### DETAILED DESCRIPTION

Provided herein are compounds that may be used as tagging agents, polymers that include the compounds, polymer compositions that include the polymers, methods for forming the compounds and polymers, and methods for monitoring a concentration of the compounds, polymers, or polymer compositions.

### Compounds

Compounds are provided herein, which may be used as fluorescent tagging monomers in polymers, including those disclosed herein. The compounds provided herein may be present as a monomer (e.g. a comonomer and/or end group) of the polymers described herein. As used herein, the phrases “tagging agent”, “tagging monomer”, and the like refer to a compound and/or monomer that is detectable at a desirable concentration (e.g., a relatively low concentration) using an analytical technique, such as fluorescence spectroscopy.

The tagging monomers provided herein, in some embodiments, exhibit a fluorescence emission maximum at about 410 nm to about 680 nm, about 410 nm to about 600 nm, about 410 nm to about 590 nm, about 410 nm to about 520 nm, about 410 nm to about 500 nm, about 440 nm to about 450 nm, about 500 nm to about 520 nm, about 550 nm to about 590 nm, about 640 nm to about 680 nm, or about 570 nm to about 600 nm, thereby providing polymer compositions or other products with a feature that may permit an amount (e.g., a concentration) of a polymer composition that includes a tagging monomers to be monitored. In some embodiments, the excitation and emission wavelengths are determined, and, therefore, may be adjusted, by selecting a particular aryl alcohol/amino aryl alcohol. The fluorescence emission may be affected by pH: for example, the keto-enol tautomers described herein may exhibit different fluorescence emissions.

The compounds, including tagging monomers, provided herein include compounds or isomers of formula (I), formula (I′), formula (I″), or formula (II). The phrases “compound of formula (I)”, “compound of formula (I′)”, “compound of formula (I″)”, “compound of formula (II)”, the term “compound” when it refers to formula (I) or (II), the term “isomer” when it refers to formula (II), and the like, as used herein, refer to and include compounds according to or isomers of, respectively, the structures of each formula, salts thereof, hydrates thereof, salt hydrates thereof, stereoisomers thereof, dehydrates thereof, and derivatives thereof. Therefore, the formulas and structures provided herein encompass and read on the formulas and structures as drawn or isomers of the formulas and structures as drawn, as well as salts, hydrates, salt hydrates, stereoisomers, dehydrates, tautomers, or derivatives of each formula and structure or isomer thereof. The “derivatives” of each formula and structure include, but are not limited, to polymers (e.g., oligomers, copolymers, etc.) formed of the compounds. The tautomers may include keto-enol tautomers.

The compounds provided herein include compounds or isomers of formula (I) or formula (II), which, as explained herein, include salts, hydrates, salt hydrates, stereoisomers, dehydrates, tautomers, and derivatives of the compounds or isomers of formula (I) and formula (II):

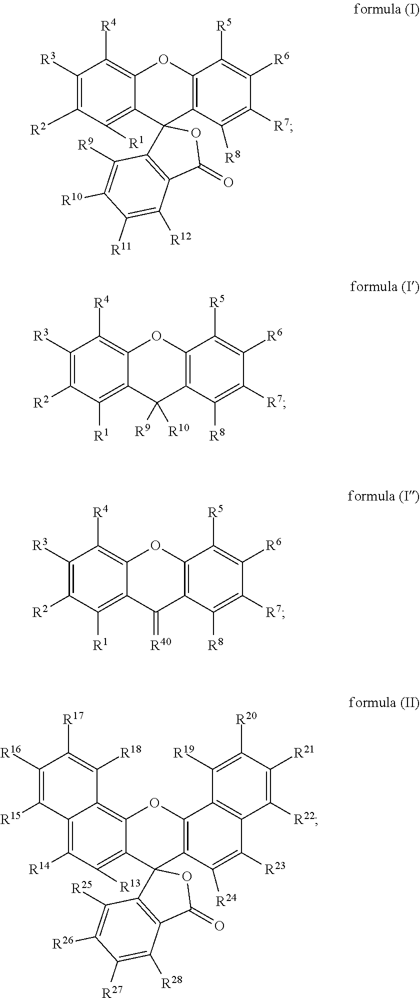

wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ are independently selected from the group consisting of hydrogen, hydroxyl, C₁-C₆ alkoxy, C₂-C₆ alkenoxy, C₂-C₆ alkynoxy, —N(R′)(R″), C₁-C₆ alkyl, C₂-C₆ alkenyl, C₂-C₆ alkynyl, and C₄-C₁₄ aryl, wherein R⁴⁰ is selected from the group consisting of C₁-C₆ alkoxy, C₂-C₆ alkenoxy, C₂-C₆ alkynoxy, —N(R′), —N(R′)(R″), C₂-C₆ alkyl, C₂-C₆ alkenyl, C₂-C₆ alkynyl, and C₄-C₁₄ aryl, wherein R′ and R″ are independently selected from the group consisting of hydrogen and C₁-C₆ alkyl, and wherein the isomers of formula (II) comprise a compound of formula (IIi), a compound of formula (IIii), a compound of formula (IIiii), or a combination thereof—

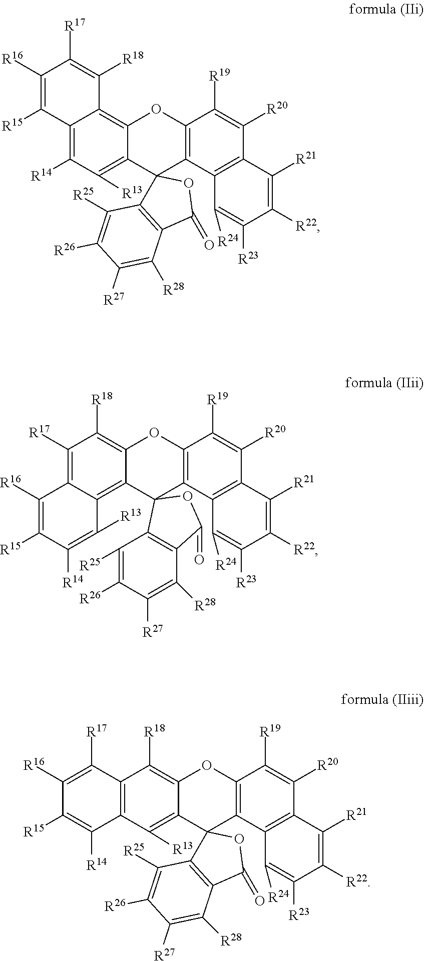

The compounds provided herein may include “R” groups (e.g., R¹, R², etc.) selected from C₁-C₆ alkoxy, C₂-C₆ alkenoxy, C₂-C₆ alkynoxy, C₁-C₆ alkyl, C₂-C₆ alkenyl, C₂-C₆ alkynyl, C₄-C₁₄ aryl, C₁-C₆ alkenyl, and the like.

Each C₁-C₆ alkoxy, C₂-C₆ alkenoxy, C₂-C₆ alkynoxy, C₁-C₆ alkyl, C₂-C₆ alkenyl, C₂-C₆ alkynyl, C₄-C₁₄ aryl, C₁-C₆ alkenyl, and the like disclosed herein, includes all substituted, unsubstituted, branched, and linear analogs or derivatives thereof, in each instance having the indicated number of carbon atoms. Examples of alkyl groups include, but are not limited to, methyl, ethyl, propyl, isopropyl, n-butyl, t-butyl, isobutyl, pentyl, hexyl, isohexyl, cyclopropyl, cyclobutyl, cyclopentyl, and cyclohexyl. Additional examples of alkyl moieties have linear, branched and/or cyclic portions. Representative alkenyl moieties include, but are not limited to, vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and 1-hexenyl. Representative alkynyl moieties include, but are not limited to, acetylenyl, propynyl, 1-butynyl, 2-butynyl, 1-pentynyl, 2-pentynyl, 3-methyl-1-butynyl, 4-pentynyl, 1-hexynyl, 2-hexynyl, and 5-hexynyl. Examples of alkoxy, alkenoxy, and alkyoxy compounds include any of the foregoing alkyl groups, alkenyl groups, or aklynyl groups that are covalently bonded to an oxygen atom. Examples of aryl moieties include, but are not limited to, anthracenyl, azulenyl, biphenyl, fluorenyl, indan, indenyl, naphthyl, phenyl, 1,2,3,4-tetrahydro-naphthalene, and the like, including substituted derivatives thereof, in each instance having from 4 to about 14 carbons. Substituted derivatives of aromatic compounds include, but are not limited to, tolyl, xylyl, mesityl, and the like, including any heteroatom substituted derivative thereof.

Each C₄-C₁₄ aryl group of the compounds provided herein may independently include (i) a single “R” substituent (for example, one of R¹, R², R³, R⁴, R⁷, or R⁸), or (ii) at least two “R” substituents on adjacent carbon atoms (for example, R¹ and R², wherein R¹ and R² are covalently bonded to each other: R² and R³, wherein R² and R³ are covalently bonded to each other; R³ and R⁴, wherein R³ and R⁴ are covalently bonded to each other; etc.). Therefore, for example, in formula (I), an unsubstituted C₆ aryl group (i.e., a phenyl) may be selected for each of R² and R³ (Structure (a)), or an unsubstituted C₄ aryl group may be selected jointly for R² and R³, thereby resulting in a 6-membered aryl ring that includes the carbon atom to which R² is covalently bonded, the carbon atom to which R³ is covalently bonded, R², and R³, wherein R² and R³ are covalently bonded to each other (Structure (b)):

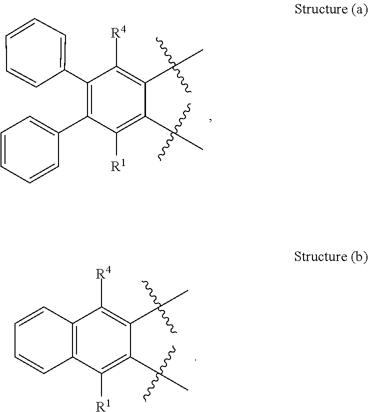

Each C₁-C₆ alkoxy, C₂-C₆ alkenoxy, C₁-C₆ alkyl, C₂-C₆ alkenyl, and/or C₁-C₆ alkenyl of the compounds provided herein may independently include (i) a single “R” substituent (for example, one of R′, R″, R¹, R², R³, R⁴, R⁷, or R⁸), or (ii) at least two “R” substituents on adjacent carbon atoms (for example, R¹ and R², wherein R¹ and R² are covalently bonded to each other: R² and R³, wherein R² and R³ are covalently bonded to each other; R³ and R⁴, wherein R³ and R⁴ are covalently bonded to each other; R′ and R², wherein R′ and R² are covalently bonded to each other, etc.). Therefore, for example, in formula (I), an unsubstituted C₆ alkyl group (i.e., a hexyl) may be selected for each of R² and R³ (Structure (a)), or an unsubstituted C₄ alkyl group may be selected jointly for R² and R³, thereby resulting in a 6-membered ring that includes the carbon atom to which R² is covalently bonded, the carbon atom to which R³ is covalently bonded, R², and R³, wherein R² and R³ are covalently bonded to each other (Structure (b)):

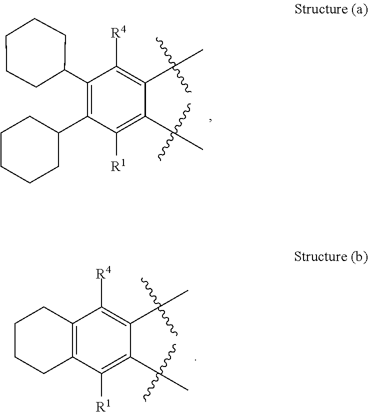

In some embodiments, any two adjacent “R” groups are not bonded to each other. For example, substituents R⁹ and R¹⁰ of formula (I′) may not be bonded to each other.

When used herein with regard to the selection of a substituent, the term “independently” indicates that (i) a substituent at a particular location may be the same or different for each molecule or monomer of a formula (e.g., (a) a compound of formula (I) may include two molecules of formula (I), with each molecule having the same or a different hydrocarbyl selected for R¹; or (b) a polymer including formula (I) may include two monomers of formula (I), with each monomer having the same or a different hydrocarbyl selected for R¹), and/or (ii) two differently labeled substituents selected from the same pool of substituents may be the same or different (e.g., R¹ and R² of a monomer may both be selected from “a C₁-C₆ alkyl”, and the C₁-C₆ alkyls selected for R¹ and R² may be the same or different).

Unless otherwise indicated, the term “substituted,” when used to describe a chemical structure or moiety, refers to a derivative of that structure or moiety wherein (i) a multi-valent non-carbon atom (e.g., oxygen, nitrogen, sulfur, phosphorus, etc.) is bonded to one or more carbon atoms of the chemical structure or moiety (e.g., a “substituted” C₄ alkyl may include, but is not limited to, diethyl ether moiety, a methyl propionate moiety, an N,N-dimethylacetamide moiety, a butoxy moiety, etc.) or (ii) one or more of its hydrogen atoms (e.g., chlorobenzene may be characterized generally as an aryl C₆ aryl “substituted” with a chlorine atom) is substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), tertiary amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-). When an “R” group (e.g., R¹) is substituted, the carbon atoms in the substituents are included in the total count of carbon atoms in the “R” group. For example, if R¹ is selected from a C₁-C₆ alkyl, and the C₁-C₆ alkyl is a propyl group substituted with a dimethylamine substituent, then R¹ is considered, in this example, to be a C₅ alkyl because there are 3 carbon atoms in the propyl group, and 2 carbon atoms in the dimethylamine substituent.

When a compound or isomer of formula (I), formula (I′), formula (I″), or formula (II) includes a stereocenter, the compounds or isomers of formula (I), formula (I′), formula (I″), or formula (II) include both of the (R) and (S) enantiomers. For example, Formula (IIiii), as depicted herein, includes both of the following stereoisomers:

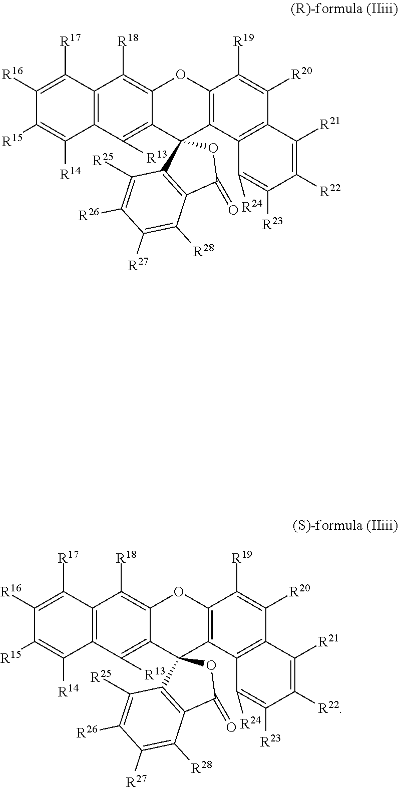

In some embodiments, R¹, R², R⁴, R⁵, R⁷, R⁸, R⁹, R¹⁰, R¹¹, and R¹² are hydrogen, and the compound is of formula (Ia):

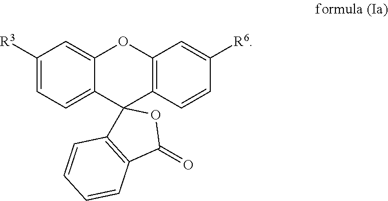

In some embodiments, the compound is of formula (I), formula (I′), formula (I″), or formula (Ia), wherein R³ and R⁶ are hydroxyl.

In some embodiments, the compound is of formula (I), formula (I′), or formula (Ia), wherein R³ and R⁶ are —N(R′)(R″). R′ and R″ may be independently selected from hydrogen and an unsubstituted C₁-C₆ alkyl, such as an unsubstituted C₂ alkyl.

In some embodiments, the compound is of formula (I′), wherein (i) R⁹ is hydrogen and R¹⁰ is methyl, (ii) at least one of R⁹ and R¹⁰ is a lactone ring, such as a C₅₊ lactone ring, or (iii) R⁹ is hydroxyl and R¹⁰ is hydrogen.

In some embodiments R¹³, R¹⁴, R¹⁵, R¹⁷, R¹⁸, R¹⁹, R²⁰, R²², R²³, R²⁴, R²⁵, R²⁶, R²⁷, and R²⁸ are hydrogen, and the compound is of formula (IIa) or an isomer thereof, as described herein:

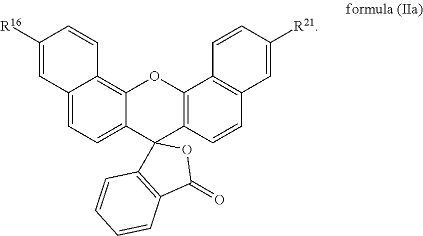

In some embodiments, R¹⁶ and R²¹ are hydroxyl in the compound or isomer of formula (II) or formula (IIa). In some embodiments, R¹⁶ and R²¹ are —N(R′)(R″) in the compound or isomer of formula (II) or formula (IIa). R′ and R″ may be an unsubstituted C₁-C₆ alkyl, such as an unsubstituted C₂ alkyl.

### Polymer Compositions

Polymer compositions are provided herein. The polymer compositions may include a copolymer, which includes a first monomer that is a tagging monomer, and at least one second monomer.

In some embodiments, the first monomer is selected from the group consisting of (a) a compound of formula (I), formula (I′), or formula (I″), and (b) a compound or isomer of formula (II), which, again, includes salts, hydrates, salt hydrates, stereoisomers, dehydrates, tautomers, or derivatives of the compounds or isomers of formulas (I) and (II). In some embodiments, the at least one second monomer includes at least one polymerizable double bond or at least one polymerizable triple bond.

In some embodiments, the copolymers are obtainable by free radical polymerization of two or more types of monomer (including 3, 4, or more different monomers) without restriction on the number of monomer units that are incorporated into the product, provided that at least one of the monomers is a first monomer (i.e., a tagging monomer) and at least one of the monomers is a second monomer, as described herein. In some embodiments, the copolymers include two or more second monomers and one or more first monomers or molecules (i.e., tagging units) as described herein.

As used herein, the terms “polymer,” “polymers,” “polymeric,” and the like are used in their ordinary sense as understood by one ordinarily skilled in the art, and thus may be used herein to refer to or describe a large molecule (or group of such molecules) that contains recurring units (i.e., monomers), including, but not limited to, oligomers, comb polymers, branched polymers, linear polymers, crosslinked polymers, star polymers, etc. Polymers may be formed in various ways, including by polymerizing monomers and/or by chemically modifying one or more recurring units of a precursor polymer. A polymer may be a “copolymer” that includes two or more different recurring units (i.e., monomers), including end groups, formed by, e.g., copolymerizing two or more different monomers (e.g., 2, 3, 4, 5, 6 or more monomers), and/or by chemically modifying one or more recurring units of a precursor polymer.

The polymers, including copolymers, provided herein are defined in terms of the monomer(s) that form the structures of the polymers. Although, in the interest of clarity, monomers are depicted in isolated, unpolymerized form herein, a person ordinarily skilled in the art will understand the structural differences between the monomers in unpolymerized and polymerized forms. For example, a person ordinarily skilled in the art will understand that an embodiment of a polymerized monomer of formula (Ia) may have the following structure or a similar structure when present as an end group in the polymers described herein:

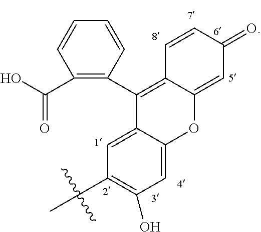 [SMILES: *c1cc2c(-c3ccccc3C(=O)O)c3ccc(=O)cc-3oc2cc1O]

Any atom of a “first monomer” may bond to another monomer, but it is believed that carbon atoms at the 2′, 4′, 5′, and 7′ positions (as depicted, for example, in the foregoing structure) have the greatest potential to react, especially with a macroradical.

The term “anti-scalant”, the phrases “scale inhibition”, “scale inhibitor” or “scale-inhibiting”, and the like generally refer to materials (e.g., monomers, polymer compositions, etc.) that may be applied (e.g., at substoichiometric levels) to interfere with crystal nucleation, growth, agglomeration, or a combination thereof. As used herein, the terms “anti-scalant”, the phrases “anti-scale agent” and “scale inhibitor”, and the like are used in their ordinary sense as understood by one skilled in the art, and thus may be used herein to refer to or describe chemical compounds or compositions, such as polymer compositions, containing such compounds, where the compounds, when added to an system, reduce or inhibit the amount of scale and/or rate of formation of scale in the system, as compared to a system that does not contain the added chemical compound or composition. In this context, the term “scale” or the phrase “mineral scale” refer to insoluble substances, such as insoluble salts, that may have a tendency to form in aqueous systems, such as boiler water, cooling water, seawater (e.g. in oil platform applications), brackish water, oilfield water, municipal treatment plant water, paper mill water, mining water, industrial treatment plant water, etc.

The phrases “treatment of scale”, “treated for scale”, “preventing or reducing scale formation”, and the like will be understood by those skilled in the art to have a broad and customary meaning that includes using the scale-inhibiting polymer compositions herein to (i) reduce an amount of scale, (ii) inhibit an amount of scale, (iii) reduce a rate of formation of scale, or (iv) a combination thereof in various systems, including aqueous systems, as compared to comparable systems that do not contain the anti-scale polymer composition.

### 1. First Monomer

The first monomer of the polymer compositions may include (a) a compound of formula (I), formula (I′), or formula (I″), or (b) a compound or isomer of formula (II), which, again, may include salts, hydrates, salt hydrates, stereoisomers, dehydrates, tautomers, or derivatives of the compounds or isomers of formulas (I), (II), or a combination thereof. Therefore, the first monomer, for example, may include a salt or salt hydrate of a compound or isomer of formula (I), formula (I′), formula (I″), or formula (II), such as a hydrochloride, dihydrochloride, sulfate, bisulfate, or gluconate salt, or hydrate thereof. As a further example, the first monomer may include a derivative of a compound or isomer of formula (I), (I′), or (II), such as a derivative formed from the addition of acid and heat to the compound or isomer of formula (I), (I′), or (II).

ii. Second Monomer

The at least one second monomer of the polymer compositions provided herein may include any monomer that includes a polymerizable moiety, such as a double bond or a triple bond. In some embodiments, the at least one second monomer is a scale inhibitor before and after polymerization, or after polymerization.

In some embodiments, the at least one second monomer includes acrylamide, an acrylate (e.g., sodium acrylate), or a combination thereof. The at least one second monomer, such as an acrylate, may include any suitable countercation (e.g., Na⁺). In some embodiments, the at least one second monomer is selected from the group consisting of allylsulfonate salts, for example sodium allylsulfonate: acrylic acid: vinyl sulfonic acid: vinyl sulfonate salts: vinyl phosphoric acid: vinyl phosphonate salts: vinylidene diphosphonic acid or salts thereof: methacrylic acid: vinyl acetate: vinyl alcohol: vinyl chloride: unsaturated mono- or di-carboxylic acids or anhydrides, such as maleic anhydride, maleic acid, fumaric acid, itaconic acid, aconitic acid, mesaconic acid, citraconic acid, crotonic acid, isocrontonic acid, angelic acid, and tiglic acid: vinyl chloride: styrene-p-sulfonic acid, or styrene sulfonates salts: acrylamido-2-methylpropanesulfonic acid (AMPS); hydroxyphosphonoacetic acid (HPA); hypophosphorus acids; acrylamides; propargyl alcohol having formula HC≡C—CH₂—OH: butyr-1,4-diol, and mixtures thereof. In some embodiments, two or more types of scale-inhibiting monomer are used as the at least one second monomer: for example, (i) sodium allylsulfonate and maleic acid, (ii) sodium allylsulfonate and maleic anhydride, (iii) sodium allylsulfonate and acrylic acid, or (iv) sodium allylsulfonate, acrylic acid, and at least one of maleic acid or maleic anhydride.

The polymer compositions provided herein generally may include any amount of at least one first monomer and any amount of at least one second monomer. In some embodiments, the first monomer is present in the copolymer at an amount of about 0.01% to about 10%, by weight, based on the weight of the copolymer. In some embodiments, the first monomer is present in the copolymer at an amount of about 0.01% to about 5%, by weight, based on the weight of the copolymer. In some embodiments, the first monomer is present in the copolymer at an amount of about 0.01% to about 2%, by weight, based on the weight of the copolymer. In some embodiments, the first monomer is present in the copolymer at an amount of about 0.01% to about 1.5%, by weight, based on the weight of the copolymer. In some embodiments, the first monomer is present in the copolymer at an amount of about 0.01% to about 1%, by weight, based on the weight of the copolymer. In some embodiments, the first monomer is present in the copolymer at an amount of about 0.01% to about 0.75%, by weight, based on the weight of the copolymer. In some embodiments, the first monomer is present in the copolymer at an amount of about 0.01% to about 0.5%, by weight, based on the weight of the copolymer. In some embodiments, the first monomer is present in the copolymer at an amount of about 0.01% to about 30%, about 0.01% to about 20%, about 0.01% to about 15%, about 0.01% to about 10%; about 0.01% to about 8%: about 0.01% to about 7%: about 0.01% to about 5%: about 0.01% to about 3%, or about 0.01% to about 2%, by weight, based on weight of the copolymer.

In some embodiments, the copolymer of a polymer composition has a weight average molecular weight (Mw) of at least 100,000 Daltons, at least 250,000 Daltons, at least 500,000 Daltons, at least 750,000 Daltons, at least 1,000,000 Daltons, at least 2,500,000 Daltons, at least 5,000,000 Daltons, at least 7,500,000 Daltons, or at least 10,000,000 Daltons. In some embodiments, the copolymer of a polymer composition has a weight average molecular weight (Mw) of about 100,000 Daltons to about 10,000,000 Daltons, about 500,000 Daltons to about 10,000,000 Daltons, or about 1,000,000 Daltons to about 10,000,000 Daltons.

In some embodiments, the polymer compositions may include one or more monomers, groups, or units, as necessary or desired, in addition to the first monomer and at least one second monomer. For example, the polymers may include one or more other groups resulting from a polymerization initiator, end-capping groups, or a combination thereof. In some embodiments, the end capping groups are derived from initiator compounds used in the polymerization of monomers.

The thermal stability of the polymer compositions may be evaluated by heating the polymer in a liquid, for example water or brine, to a temperature, for example, of about 80° C., about 90° C., about 100° C., about 110° C., about 120° C., or about 130° C., and keeping polymer composition in the liquid at that temperature for a period of time, for example, about one week.

In some embodiments, the polymer compositions, including the copolymers provided herein, have a thermal stability such that when a polymer composition is kept at a temperature of about 80° C. in water or brine for about one week, there is less than about a 15%, about a 10%, about a 5%, about a 4%, or about a 3% decrease in emission intensity. In some embodiments, the polymer compositions, including the copolymers provided herein, have a thermal stability such that when a polymer composition is kept at a temperature of about 130° C. in water or brine for about one week, there is less than about a 15%, about a 10%, about a 5%, about a 4%, or about a 3% decrease in emission intensity. In some embodiments, the water is at a pH of about 7 to about 8. In some embodiments, the brine is natural brine or synthetic brine. In some embodiments, the polymer compositions have a thermal stability such that when a polymer composition is kept at a temperature of about 80° C. in water for about one week, there is less than about a 10%, about a 5%, about a 4%, or about a 3% decrease in emission intensity. In some embodiments, the polymer compositions have a thermal stability such that when a polymer composition is kept at a temperature of about 130° C. in water for about one week, there is less than about a 15%, about a 10% or about a 5% decrease in emission intensity. In some embodiments, the polymer compositions have a thermal stability such that when a polymer composition is kept at a temperature of about 130° C. in water at about pH 8 for about one week, there is less than about a 15%, about a 13%, or about a 10% decrease in emission intensity. In some embodiments, the polymer compositions have a thermal stability such that when a polymer composition is kept at a temperature of about 130° C. in brine for about one week, there is less than about a 20%, about a 15% or about a 10% decrease in emission intensity.

The polymer composition may optionally include one or more additional ingredients, as necessary or desired, such as those described herein, which include water, salts, oils, surfactants, pH adjusting agents (such as acids, bases and buffers), colorants, flow modifiers, other water treatment agents, etc. In some embodiments, the polymer composition consists essentially of a copolymer that includes a first monomer and at least one second monomer, as described herein. When the polymer composition consists essentially of a copolymer that includes a first monomer and at least one second monomer, the polymer composition may include one or more of the foregoing “additional ingredients” and the following “[e]xemplary fluids”, because the “additional ingredients” and “[e]xemplary fluids” are non-limiting examples of components that do not materially affect the basic and novel characteristic(s) of the polymer compositions.

In some embodiments, the polymer compositions include (i) a copolymer of a first monomer and at least one second monomer, and (ii) a fluid. Exemplary fluids include those that may be in or intended for industrial water systems or process systems, such as boilers, cooling systems, cooling towers, desalination plants, geothermal power production, irrigation systems, mineral ore extraction systems, paper pulping or manufacturing systems, membrane systems, etc. Other exemplary fluids include fluids for use in the oil industry, such as those for use in the treatment of water injection systems, subsea flow lines, topside production equipment and “down-hole” to control scaling in and around the production well-bore. In some embodiments, the fluid includes tailings, such as oil and/or sand tailings. The polymer compositions may be used for polymer flooding.

In some embodiments, the polymer compositions include an aqueous composition or a water-based fluid, for example a seawater-based fluid. Other fluids, however, are envisioned.

In some embodiments, the polymer compositions include a glycol or glycol ether based solvent.

The polymer compositions described herein may or may not include a fluid. In some embodiments, the polymer compositions described herein include dry polyacrylamide (DPAM) polymer compositions.

In some embodiments, the polymer compositions include a copolymer of a first monomer and at least one second monomer, as described herein, and, optionally, one or more additional polymers. The one or more additional polymers may include a tagging agent, and the fluorescence emission of the tagging agent may differ from the fluorescence emission of the first monomer of the copolymer.

In some embodiments, the polymer composition includes one or more copolymers, as described herein, in combination with one or more additional ingredients, such as anionic surfactants (e.g. C₁₀₋₂₀ alkyl benzene sulfonates, C₁₀₋₂₀ olefin sulfonates, C₁₀₋₂₀ alkyl sulfates, C₁₀₋₂₀ alkyl 1 to 25 mole ether sulfates, C₁₀₋₂₀ paraffin sulfonates, C₁₀₋₂₀ soaps, C₁₀₋₂₀ alkyl phenol sulfates, sulfosuccinates, sulfosuccinamates, lignin sulfonates, fatty ester sulfonates, C₁₀₋₂₀ alkyl phenyl ether sulfates, C₁₀₋₂₀ alkyl ethanolamide sulfates, C₁₀₋₂₀ alpha sulfo fatty acid salts, C₁₀₋₂₀ acyl sarcosinates, isethionates, C₁₀₋₂₀ acyl taurides, C₁₀₋₂₀ alkyl hydrogen phosphates), non-ionic surfactants (e.g. ethoxylated and/or propoxylated C₁₀₋₂₀ alcohols, ethoxylated and/or propoxylated C₁₀₋₂₀ carboxylic acids, alkanolamides, amine oxides, and/or C₁₀₋₂₀ acyl sorbitan and/or glyceryl ethoxylates), amphoteric surfactants (e.g. betaines, sulfobetaines, and/or quaterised imidazolines), and/or cationic surfactants (e.g. benzalkonium salts, C₁₀₋₂₀ alkyl trimethyl ammonium salts, and/or C₁₀₋₂₀ alkyl trimethyl): sequestrants; chelating agents: corrosion inhibitors (e.g., imidazoline and quaterantry ammonium salts); and/or other threshold agents (e.g. polymers such as aminometholine phosphonate polymers, polyacrylic acid, or non polymeric agents such as sodium tripolyphosphate, sodium ethylenediamine tetracetate, sodium nitrilo triacetate, tetra potassium pyrophosphate, acetodiphosphonic acid and its salts, ammonium trismethylene phosphonic acid and its salts, ethylenediamine tetrakis(methylene phosphonic) acid and its salts, diethylenetriamine pentakis(methylene phosphonic) acid and its salts); tolyltriazole and mixtures of nitrate, benzqate, HHP and/or PTCB): hydrate inhibitors (e.g., methanol); cinetic inhibitors such as anti-agglomeration agents: biocides (e.g. tetrakis(hydroxymethyl) phosphonium salts, formaldehyde, glutaraldehyde, DENPA, bromopol isothiazoronal): oxidising biocides and/or bleaches (e.g. chlorine, chlorine dioxide, hydrogen peroxide, sodium perborate): foam controlling agents, such as silicone antifoams: oxygen scavengers such as hydrazines and/or hydroxylamines: pH controlling and/or buffering agents, such as amines, borates, citrates and/or acetates: chromium salts: zinc salts; asphaltene inhibitors: wax inhibitors; demulsifiers: other scale inhibitors; and/or other water treatment agents such as polymeric dispersants and coagulants including polymaleic, polyacrylic and polyvinylsulfonic acids and their salts, starches and/or carboxy methyl cellulose, and/or molybdates.

In some embodiments, the polymer composition includes two or more copolymers. When two or more copolymers are present, each copolymer may include a different first monomer, and each of the different first monomers or molecules may exhibit a different fluorescence emission. For example, a polymer composition may include (i) a first copolymer including a first monomer, which has a fluorescence emission maximum of about 500 to about 520 nm, (ii) a second copolymer including a first monomer, which has a fluorescence emission maximum of about 550 nm to about 590 nm, (iii) a third copolymer including a first monomer, which has a fluorescence emission maximum of about 640 nm to about 680 nm, or (iv) a combination thereof. Such a polymer composition also may include an additional copolymer including a first monomer, which has a fluorescence emission maximum of about 570 nm to about 600 nm. The differences in fluorescence emission maximum may permit the methods described herein to be used to determine an amount of each copolymer present in a fluid or system, the differences between the amounts of each copolymer in a fluid or system, or a combination thereof.

In some embodiments, the polymer compositions include about 5% to about 95%, by weight, of a copolymer of a first monomer and at least one second monomer, as described herein, and about 5% to about 90%, by weight, of one or more of any of the additional ingredients described herein, based on the total weight of a polymer composition.

A copolymer of at least one first monomer and at least one second monomer may be combined with water using any suitable method. For example, a copolymer may be dissolved, suspended, dispersed, or emulsified in water. The amount of water in an aqueous polymer composition may vary, as necessary or desired. For example, an aqueous polymer composition may include about 20% to about 80%, by weight, of a copolymer of a first monomer and a second monomer, as described herein, based on the total weight of the aqueous polymer composition.

In some embodiments, the pH of a polymer composition may be such that the acidic functionalities of a copolymer, as described herein, are neutralized. For example, the composition may be neutralized by adjusting the pH of the composition to a pH in a range of about 2 to about 13. In some embodiments, the copolymer is an anionic copolymer. In some embodiments, the copolymer is a neutral copolymer. In some embodiments, the copolymer is a cationic copolymer.

### Methods of Synthesis

The compounds or isomers of formula (I), (I′), or (II) may be synthesized with any technique, including those provided herein.

In some embodiments, the compounds or isomers of formula (I), (I′), or (II) are formed via a condensation reaction. The condensation reaction may include contacting an aryl alcohol, a condensation catalyst, and a compound according to formula (A) to form the condensation product. The condensation product may include a compound or isomer of formula (I), (I′), or (II), which, as explained herein, includes the salts, hydrates, salt hydrates, stereoisomers, dehydrates, tautomers, and derivatives of the compounds or isomers of formula (I), (I′), or (II).

The compound according to Formula (A) has the following structure:

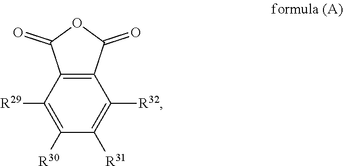

wherein R²⁹, R³⁰, R³¹, and R³² are independently selected from the group consisting of hydrogen, hydroxyl, C₁-C₆ alkoxy, C₂-C₆ alkenoxy, C₂-C₆ alkynoxy, —N(R′)(R″), C₁-C₆ alkyl, C₂-C₆ alkenyl, C₂-C₆ alkynyl, and C₄-C₁₄ aryl.

In some embodiments, R²⁹, R³⁰, R³¹, and R³² are hydrogen.

As used herein, the phrase “aryl alcohol” generally refers to a compound that includes (i) an aryl moiety, and (ii) at least one hydroxyl moiety. In some embodiments, the aryl alcohol includes (i) an aryl moiety, and (ii) two hydroxyl moieties. In some embodiments, the aryl alcohol is resorcinol. In some embodiments, the aryl alcohol is 1,6-dihydroxynaphthalene. The aryl alcohol, however, may include any compound that is capable of forming a compound or isomer of formula (I), (I′), or (II). For example, when the methods provided herein are used to produce a compound or isomer of formula (I), the aryl alcohol may be of the following formula:

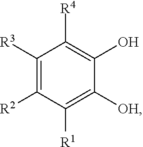

wherein R¹, R², R³, and R⁴ are as defined herein.

The contacting of the aryl alcohol, the condensation catalyst, and the compound of formula (A) may occur at any temperature and/or pressure that is effective to form the condensation product. In some embodiments, the contacting of the aryl alcohol, the condensation catalyst, and the compound of formula (A) occurs at a temperature of about 50° C. to about 150° C., about 75° C. to about 150° C., about 100° C. to about 150° C., or about 100° C. to about 125° C. In some embodiments, the contacting of the aryl alcohol, the condensation catalyst, and the compound of formula (A) occurs at ambient pressure, and a temperature of about 50° C. to about 150° C., about 75° C. to about 150° C., about 100° C. to about 150° C., or about 100° C. to about 125° C.

The condensation catalyst may include any catalyst capable of effecting the condensation of the aryl alcohol and the compound of formula (A). In some embodiments, the condensation catalyst is a Lewis acid. Non-limiting examples of Lewis acids include ZnCl₂, FeCl₃, AlCl₃, and BCl₃. In some embodiments, the condensation catalyst is a sulfonic acid. The sulfonic acid may include an C₁-C₆ alkyl sulfonic acid, a C₅-C₁₄ aryl sulfonic acid, or a combination thereof. In some embodiments, the C₁-C₆ alkyl sulfonic acid is methanesulfonic acid (MeSO₃H). In some embodiments, the C₅-C₁₄ aryl sulfonic acid is p-toluenesulfonic acid.

Also provided herein are methods of polymerizing a compound or isomer of formula (I), (I′), or (II). In some embodiments, the methods include contacting a compound or isomer of formula (I), (I′), or (II)(e.g., a condensation product of the foregoing methods) with at least one second monomer to form a copolymer, wherein the at least one second monomer includes a polymerizable double bond or triple bond. The at least one second monomer may be contacted with an amount of the condensation product effective to produce a copolymer that includes a desirable amount of the condensation product as described herein, for example, any of the amounts recited herein, such as about 0.01% to about 5%, or about 0.01% to about 2%, by weight, based on the weight of the copolymer.

The at least one second monomer generally may include any monomer that is polymerizable due to the presence of a polymerizable double bond or triple bond. The phrases “polymerizable double bond”, “polymerizable triple bond”, and the like refer to bonds that may react with a functional group of at least one other monomer (e.g., under conditions described herein) to form a polymer. In some embodiments, the at least one second monomer includes sodium acrylate, acrylamide, or a combination thereof.

The polymer compositions provided herein generally may be prepared by any polymerization method. For example, a free-radical polymerization method may be employed. Other exemplary methods include aqueous bulk/dispersion polymerization, solution polymerization, or emulsion polymerization. In some embodiments, the polymerization process is a solution polymerization, wherein water is charged to a reaction vessel fitted with a mechanical stirrer and water condenser, and heated to a temperature within a range of about 45° C. to about 150° C., or about 45° C. to about 110° C. One or more polymerization initiators may be added to the reactor. The choice of initiator may inform the temperature at which the reaction is performed. A first monomer may be added to the reactor, added to a monomer feed or fed separately. A monomer feed(s), soluble initiator feed, and optionally a chain transfer reagent feed may be added to a vessel at a predetermined time or over time.

In some embodiments, the polymerization of monomers, including at least one first monomer and at least one second monomer, is achieved in the presence of one or more polymerization initiators including, but not limited to, inorganic peroxides, for example ammonium persulfate (APS), hydroxymethanesulfinic acid monosodium salt dehydrate, potassium persulfate, and sodium persulfate: organic peroxides, for example tert-butyl hydroperoxide (TBHP), tert-butyl peracetate, cumene hydroperoxide, 2,5-Di(tert-butylperoxy)-2,5-dimethyl-3-hexyne, dicumyl peroxide, 2,5-bis(tert-butylperoxy)-2,5-dimethylhexane, 2,4-pentanedione peroxide, 1,1-bis(tert-butylperoxy)-3,3,5-trimethylcyclohexane, 1,1-bis(tert-butylperoxy)cyclohexane, 1,1-bis(tert-amylperoxy) cyclohexane, benzoyl peroxide, 2-butanone peroxide, tert-butyl peroxide, lauroyl peroxide, tert-butyl peroxy benzoate, and tert-butylperoxy 2-ethylhexyl carbonate: azo compounds, for example azobisisobutyronitrile (AIBN), 4,4′-azobis(4-cyanovaleric acid), 1, l′-azobis(cyclohexanecarbonitrile), 2,2′-azobis(2-methylpropionamidine) dihydrochloride, and 2,2′-azobis(2-methylpropionitrile): tetrakis(hydroxy methyl) phosphonium sulfate (THPS); cerium ammonium nitrate: perchlorates: triphenylphosphine; and the like, and compositions or mixtures including one or more of these initiators. In some embodiments, the initiator is selected from the group consisting of ammonium persulfate, tert-butyl hydroperoxide, and 4,4′-azobis(4-cyanovaleric acid).

Polymerization initiators generally may be used at an amount of about 0.01% to about 10%, by weight, based on the total weight of the monomers. Polymerization initiators may be used in conjunction with heat to initiate polymerization of monomers. In some embodiments, two or more initiators are used: for example, an inorganic peroxide and an organic peroxide. In some embodiments, ammonium persulfate (APS) and an organic peroxide are used to initiate polymerization. The initiator or initiators used to achieve polymerization may affect the physical properties of the resulting polymer. The initiator or initiators may be added to a polymerization reaction mixture, for example, at the start of the reaction, at various times during the polymerization, and/or gradually over time, e.g., over several minutes or hours. If two or more initiators are used, then the initiators may be dosed simultaneously or sequentially during polymerization. In some embodiments, one initiator is dosed at the start of polymerization, at various times during polymerization, and/or gradually over time, and a different initiator is used at later stages the polymerization.

### Methods for Determining Polymer Concentration

Methods for determining polymer concentration are provided. In some embodiments, the methods include providing a system that includes a fluid in circulation, wherein the fluid includes a polymer composition as described herein: measuring with an analytical technique an amount of the first monomer in the system or the fluid to determine an amount of the polymer composition in the system or the fluid, wherein the measuring is performed periodically or continuously; and optionally (i) adding an additional amount of the polymer composition to the system or the fluid if the amount of the polymer composition in the system or the fluid is less than a predetermined value, or (ii) removing a portion of the polymer composition from the system or the fluid if the amount of the polymer composition in the system or the fluid is greater than the predetermined value.

In some embodiments, the fluid in circulation includes tailings, such as oil and/or sand tailings. In some embodiments, the system also includes a centrifuge. In some embodiments, the system also includes a centrifuge, the fluid in circulation also includes tailings, and the providing of the system includes processing the fluid in circulation with the centrifuge to produce an amount of retained solids and a centrate. The measuring of the amount of the polymer composition in the fluid may permit a user to maintain in the system a desired amount of the polymer composition for one or more reasons, such as separating a certain percentage of solids with a centrifuge or other device.

As used herein, the phrase “amount of the first monomer”, the phrase “amount of the polymer composition”, and the like refer to and include (i) an actual numerical amount (e.g., X grams) of the first monomer or the polymer composition, respectively, in a fluid or system, or (ii) a concentration (e.g., X ppm) of the first monomer or polymer composition, respectively, in a fluid or system.

In some embodiments, the methods include (a) adding to a system or fluid a predetermined amount of a polymer composition as described herein: (b) periodically or continuously measuring the amount of tagging units (i.e., first monomer) in the system or fluid to determine an amount of the polymer composition in the system or fluid; and (c) periodically or continuously further adding more or removing a portion of the polymer composition to or from the system or fluid when the measured amount of tagging units (i.e., first monomer) is less than or greater than, respectively, a predetermined value.

In some embodiments, when the measured amount of a tagging first monomer (or the polymer composition) in a system or fluid being treated is less than a predetermined value, more polymer composition may be added to the system or fluid. The predetermined value of polymer may be any amount necessary or desired for the particular system or fluid being treated.

The polymer compositions herein may be detected (e.g., measured) by any appropriate method, including, but not limited to, fluorometry. In some embodiments, the polymer compositions are detected with a fixed wavelength fluorometer. Detection may be at the polymer maxima excitation (ex) and emission (em) wavelengths. These wavelengths may be determined using a scanning fluorometer in scanning mode. The level of fluorescence may be determined by the Beer-Lambert Law. For example, concentrations may be assigned by comparison of the emission intensity of a polymer composition sample with a calibration plot obtained from polymer samples of a known concentration. Any detection method which utilizes the fluorescence properties of the polymer compositions, particularly the first monomer, may be used, as necessary or desired.

The constituents of a liquid, e.g., water, may be considered when determining the proper application of the polymer compositions provided herein, as some of the constituents may have natural fluorescence properties (for example, certain polycyclic hydrocarbons) that may interfere with the detection of the tagging units (i.e., first monomer) of the polymer compositions. The chemical properties of produced water may vary considerably depending on the location and the geological formation of an oil field, as well as the type of hydrocarbons being produced. Produced water properties also may vary throughout the lifetime of a reservoir. Most of the naturally fluorescent properties of produced waters typically originate from hydrocarbon residues or other production chemicals in the produced waters. Even though the amount of these species might be minimal, fluorescence can detect these species at very low ppm levels.

In some embodiments, the polymer compositions can be used in combination or alternation with other tagged polymers or tagged polymer compositions, and in particular, with other tagged polymers or tagged polymer compositions including fluorescent moieties that have excitation and/or emission that are different from those of the polymer compositions described herein. The use of the polymer compositions described herein with other tagged polymers or tagged polymer compositions may be referred to as a multi-tagged system. A multi-tagged system could be used, for example, to allow an operator to monitor two different polymers in a system being treated with a polymer composition as provided herein. An example of such a system would include one in which more than one well is drilled and the oil from all wells is collected from one central location. A different polymer composition and/or other tagged polymer may be introduced to each well. From a single sample collected at the central location, an operator may determine which specific well requires more polymer composition and/or other tagged polymer by monitoring the presence and/or concentration of each tagged polymer.

In some embodiments, the polymer compositions are combined in a multi-tagged system with one or more polymers having a different tagging unit than the polymer compositions. Exemplary tagging units are described, for example, in one or more of the following references (each of which is incorporated herein by reference): U.S. Pat. Nos. 7,703,516; 7,943,058; 9,902,904: EP 1 636 142: EP 1 639 228; U.S. Patent Application Publication. No. 2012/0032093.

As used herein, the phrase “effective detection amount” refers to an amount of tagging units (i.e., first monomer) sufficient to provide suitable detection in a particular application. In some embodiments, the polymer composition includes an effective detection amount of tagging units (i.e., first monomer). In some embodiments, an effective detection amount of the first monomer in the polymer compositions is about 0.01% to about 30%, about 0.01% to about 20%, about 0.01% to about 15%, about 0.01% to about 10%; about 0.01% to about 8%: about 0.01% to about 7%; about 0.01% to about 5%; about 0.01% to about 3%, about 0.01% to about 2%, about 0.01% to about 1.5%, about 0.01% to about 1%, about 0.01% to about 0.75%, or about 0.01% to about 0.5%, by weight, based on the total weight of the polymer composition. An effective detection amount may be achieved by the amount of first monomer that is polymerized with an at least one second monomer to form a copolymer, an amount of unpolymerized first monomer added to a polymer composition, or a combination thereof.

The compounds and polymer compositions described herein may be used in a variety of systems, including aqueous systems. Non-limiting examples of such systems include boiler water, cooling water, seawater (e.g., in oil platform applications), brackish water, oilfield water (e.g., topside and/or downhole), municipal treatment plant water, and industrial treatment plant water. The amount of polymer composition that is effective to achieve a desired purpose in a particular aqueous system may be determined by routine experimentation in light of the guidance provided herein. The amount of polymer composition added to the aqueous system may vary over a relatively broad range, depending on the nature of the aqueous system and the type of scale. For example, the amount of polymer added to the aqueous system may be in the range of about 0.1 part per million to about 50,000 parts per million, about 0.1 part per million to about 25,000 parts per million, about 0.1 part per million to about 10,000 parts per million, about 0.1 part per million to about 1,000 parts per million, about 0.1 part per million to about 500 parts per million, or about 100 parts per million to about 200 parts per million, based on the capacity of the aqueous system.

For example, the polymer compositions and methods may be used in systems and fluids, such as oilfield injection and production waters, including topside, downhole and rock formation squeeze applications at the well site. In oilfield injection and production waters, scale formation can constrict injection lines, flow lines, and tubing strings. Without wishing to be limited by any particular theory, embodiments of the polymer compositions provided herein can modify the crystal growth of nucleating scale particles, thereby interrupting and delaying crystal growth. Embodiments of the polymer compositions also or alternatively may sequester metal ions, making them unavailable for ion pairing with anions, thereby preventing precipitation of insoluble scale.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of various embodiments, applicants in no way disclaim these technical aspects, and it is contemplated that the present disclosure may encompass one or more of the conventional technical aspects discussed herein.

The present disclosure may address one or more of the problems and deficiencies of known methods and processes. However, it is contemplated that various embodiments may prove useful in addressing other problems and deficiencies in a number of technical areas. Therefore, the present disclosure should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions: or is known to be relevant to an attempt to solve any problem with which this specification is concerned.

In the descriptions provided herein, the terms “includes,” “is,” “containing,” “having,” and “comprises” are used in an open-ended fashion, and thus should be interpreted to mean “including, but not limited to.” When compositions, systems, or methods are claimed or described in terms of “comprising” various steps or components, the compositions, systems, or methods can also “consist essentially of” or “consist of” the various steps or components, unless stated otherwise.

The terms “a,” “an,” and “the” are intended to include plural alternatives, e.g., at least one. For instance, the disclosure of “a first monomer”, “a polymer composition”, and the like, is meant to encompass one, or mixtures or combinations of more than one first monomer, polymer composition, and the like, unless otherwise specified.

Various numerical ranges may be disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. Moreover, all numerical end points of ranges disclosed herein are approximate. As a representative example, Applicant discloses, in some embodiments, the compounds exhibit fluorescence emission maxima at about 490 nm to about 500 nm. This range should be interpreted as encompassing emission maxima of about 490 nm and about 500 nm, and further encompasses “about” each of 491 nm, 492 nm, 493 nm, 494 nm, 495 nm, 496 nm, 497 nm, 498 nm, and 499 nm, including any ranges and sub-ranges between any of these values.

As used herein, the term “about” means plus or minus 10% of the numerical value of the number with which it is being used.

### EMBODIMENTS

The following is a non-limiting list of embodiments of the compositions and methods described herein:

Embodiment 1. A polymer composition comprising a copolymer comprising (i) a first monomer comprising (a) a compound of formula (I), formula (I′), or formula (I″), or (b) a compound or an isomer of formula (II), wherein the first monomer is a tagging monomer, and (ii) at least one second monomer comprising at least one polymerizable double bond or at least one polymerizable triple bond:

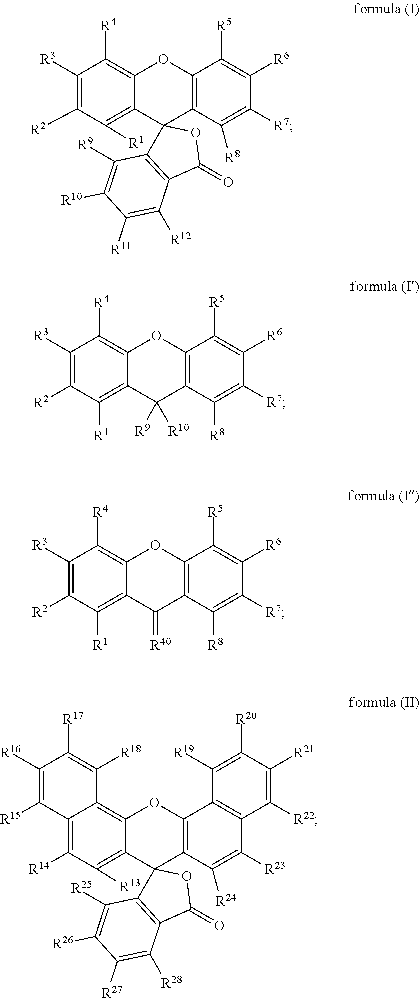

wherein R¹, R², R³, R⁴, R⁵, R⁶, R⁷, R⁸, R′, R¹⁰, R¹¹, R¹², R¹³, R¹⁴, R¹⁵, R¹⁶, R¹⁷, R 18, R¹⁹, R²⁰, R²¹, R²², R²³, R²⁴, R²⁵, R²⁶, R²⁷, and R²⁸ are independently selected from the group consisting of hydrogen, hydroxyl, C₁-C₆ alkoxy, C₂-C₆ alkenoxy, C₂-C₆ alkynoxy, —N(R′)(R″), C₁-C₆ alkyl, C₂-C₆ alkenyl, C₂-C₆ alkynyl, and C₄-C₁₄ aryl, wherein R⁴⁰ is selected from the group consisting of C₁-C₆ alkoxy, C₂-C₆ alkenoxy, C₂-C₆ alkynoxy, —N(R′), —N(R′)(R″), C₁-C₆ alkyl, such as C₂-C₆ alkyl, C₂-C₆ alkenyl, C₂-C₆ alkynyl, and C₄-C₁₄ aryl, wherein R′ and R″ are independently selected from the group consisting of hydrogen and C₁-C₆ alkyl, and wherein the isomers of Formula (II) comprise a compound of Formula (IIi), a compound of Formula (IIii), a compound of Formula (IIiii), or a combination thereof—

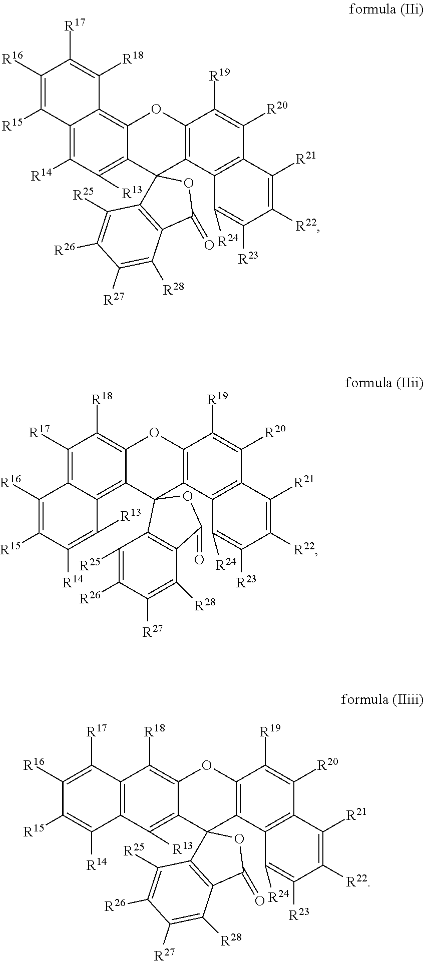

Embodiment 2. The polymer composition of Embodiment 1, wherein the copolymer has a weight average molecular weight (Mw) of at least 1,000,000 Da, or at least 10,000,000 Da.

Embodiment 3. The polymer composition of Embodiment 1, wherein the copolymer has a weight average molecular weight (Mw) of at least 100,000 Daltons, at least 250,000 Daltons, at least 500,000 Daltons, at least 750,000 Daltons, at least 1,000,000 Daltons, at least 2,500,000 Daltons, at least 5,000,000 Daltons, at least 7,500,000 Daltons, or at least 10,000,000 Daltons; or wherein the copolymer has a weight average molecular weight (Mw) of about 100,000 Daltons to about 10,000,000 Daltons, about 500,000 Daltons to about 10,000,000 Daltons, or about 1,000,000 Daltons to about 10,000,000 Daltons.

Embodiment 4. The polymer composition of any one of Embodiments 1 to 3, wherein the first monomer is present in the copolymer at an amount of about 0.01% to about 10%, about 0.01% to about 5%, about 0.01% to about 2%, about 0.01% to about 1.5%, about 0.01% to about 1%, about 0.01% to about 0.75%, or about 0.01% to about 0.5%, by weight, based on the weight of the copolymer; or wherein the first monomer is present in the copolymer at an amount of about 0.01% to about 30%, about 0.01% to about 20%, about 0.01% to about 15%, about 0.01% to about 10%, about 0.01% to about 8%, about 0.01% to about 7%, about 0.01% to about 5%, about 0.01% to about 3%, or about 0.01% to about 2%, by weight, based on weight of the copolymer.

Embodiment 5. The polymer composition of any one of Embodiments 1 to 4, wherein the at least one second monomer comprises acrylamide, sodium acrylate, or a combination thereof: or wherein the at least one second monomer is selected from the group consisting of allylsulfonate salts, for example sodium allylsulfonate; acrylic acid; vinyl sulfonic acid; vinyl sulfonate salts: vinyl phosphoric acid: vinyl phosphonate salts: vinylidene diphosphonic acid or salts thereof: methacrylic acid: vinyl acetate: vinyl alcohol: vinyl chloride: unsaturated mono- or di-carboxylic acids or anhydrides, such as maleic anhydride, maleic acid, fumaric acid, itaconic acid, aconitic acid, mesaconic acid, citraconic acid, crotonic acid, isocrontonic acid, angelic acid, and tiglic acid: vinyl chloride: styrene-p-sulfonic acid, or styrene sulfonates salts: acrylamido-2-methylpropanesulfonic acid (AMPS);

hydroxyphosphonoacetic acid (HPA); hypophosphorus acids; acrylamides; propargyl alcohol having formula HC≡C—CH₂—OH: butyr-1,4-diol, and mixtures thereof; or wherein two or more types of scale-inhibiting monomer are used as the at least one second monomer: for example, (i) sodium allylsulfonate and maleic acid, (ii) sodium allylsulfonate and maleic anhydride, (iii) sodium allylsulfonate and acrylic acid, or (iv) sodium allylsulfonate, acrylic acid, and at least one of maleic acid or maleic anhydride.

Embodiment 6. The polymer composition of any one of Embodiments 1 to 5, wherein the first monomer comprises the compound of formula (I), wherein R¹, R², R⁴, R⁵, R⁷, R⁸, R⁹, R¹⁰. R¹¹, and R¹² are hydrogen, or formula (I′) or (I″), wherein R¹, R², R⁴, R⁵, R⁷, and R⁸ are hydrogen.

Embodiment 7. The polymer composition of any one of Embodiments 1 to 6, wherein R³ and R⁶ are hydroxyl.

Embodiment 8. The polymer composition of any one of Embodiments 1 to 6, wherein R³ and R⁶ are —N(R′)(R″).

Embodiment 9. The polymer composition of Embodiment 8, wherein R′ and R″ are independently selected from hydrogen and an unsubstituted C₁-C₆ alkyl, such as an unsubstituted C₂ alkyl.

Embodiment 10. The polymer composition of any one of Embodiments 1 to 9, wherein the first monomer comprises the compound or isomer of formula (II), wherein R¹³, R¹⁴. R¹⁵. R¹⁷, R¹⁸, R¹⁹, R²⁰, R²², R²³, R²⁴, R²⁵, R²⁶, R²⁷, and R²⁸ are hydrogen.

Embodiment 11. The polymer composition of any one of Embodiments 1 to 10, wherein the first monomer comprises the compound or isomer of formula (II), wherein R¹⁶ and R²¹ are hydroxyl.

Embodiment 12. The polymer composition of any one of Embodiments 1 to 10, wherein R¹⁶ and R²¹ are —N(R)(R″).

Embodiment 13. The polymer composition of Embodiment 12, wherein R′ and R″ are independently selected from hydrogen and an unsubstituted C₁-C₆ alkyl, such as an unsubstituted C₂ alkyl.

Embodiment 14. The polymer composition of any one of Embodiments 1 to 13, wherein R¹³, R¹⁴, R¹⁵, R¹⁷, R¹⁸, R¹⁹, R²⁰, R²², R²³, R²⁴, R²⁵, R²⁶, R²⁷, and R²⁸ are hydrogen, and the compound is of formula (IIa) or an isomer thereof, as described herein:

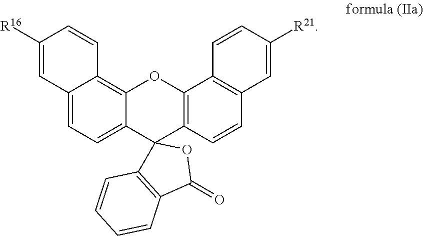

Embodiment 15. The polymer composition of Embodiment 14, wherein R¹⁶ and R²¹ are hydroxyl in the compound or isomer of formula (II) or formula (IIa); or R¹⁶ and R²¹ are —N(R′)(R″) in the compound or isomer of formula (II) or formula (IIa), wherein R′ and R″ may be an unsubstituted C₁-C₆ alkyl, such as an unsubstituted C₂ alkyl.

Embodiment 16. The polymer composition of any one of Embodiments 1 to 15, wherein the copolymer is an anionic copolymer.

Embodiment 17. The polymer composition of any one of Embodiments 1 to 16, wherein the first monomer is a comonomer and/or end group of the copolymer.

Embodiment 18. The polymer composition of any one of Embodiments 1 to 17, wherein the first monomer exhibits a fluorescence emission maximum at about 410 nm to about 680 nm, about 410 nm to about 600 nm, about 410 nm to about 590 nm, about 410 nm to about 520 nm, about 410 nm to about 500 nm, about 440 nm to about 450 nm, about 500 nm to about 520 nm, about 550 nm to about 590 nm, about 640 nm to about 680 nm, or about 570 nm to about 600 nm.

Embodiment 19. The polymer composition of any one of Embodiments 1 to 18, wherein the polymer composition or copolymer has a thermal stability such that when a polymer composition or copolymer is kept at a temperature of about 80° C. in water or brine for about one week, there is less than about a 15%, about a 10%, about a 5%, about a 4%, or about a 3% decrease in emission intensity.

Embodiment 20. The polymer composition of any one of Embodiments 1 to 19, wherein the polymer composition or copolymer has a thermal stability such that when the polymer composition or copolymer is kept at a temperature of about 130° C. or about 80° C. in water or brine for about one week, there is less than about a 15%, about a 10%, about a 5%, about a 4%, or about a 3% decrease in emission intensity: wherein, optionally, the water is at a pH of about 7 to about 8.

Embodiment 21. The polymer composition of any one of Embodiments 1 to 20, wherein the polymer composition comprises one or more additional ingredients selected from the group consisting of water, salts, oils, surfactants, pH adjusting agents (such as acids, bases and buffers), colorants, flow modifiers, and other water treatment agents.

Embodiment 22. The polymer composition of any one of Embodiments 1 to 21, wherein the polymer composition consists essentially of the copolymer that includes a first monomer and at least one second monomer.

Embodiment 23. The polymer composition of any one of Embodiments 1 to 22, wherein the polymer compositions include (i) the copolymer of a first monomer and at least one second monomer, and (ii) a fluid.

Embodiment 24. The polymer composition of Embodiment 23, wherein the fluid includes those that may be in or intended for (i) industrial water systems or process systems, such as boilers, cooling systems, cooling towers, desalination plants, geothermal power production, irrigation systems, mineral ore extraction systems, paper pulping or manufacturing systems, membrane systems, etc., (ii) use in the oil industry, such as those for use in the treatment of water injection systems, subsea flow lines, topside production equipment and “down-hole” to control scaling in and around the production well-bore.

Embodiment 25. The polymer composition of Embodiment 23 or 24, wherein the fluid includes tailings, such as oil and/or sand tailings.

Embodiment 26. The polymer composition of any one of Embodiments 1 to 25, wherein the polymer composition is used for- or configured for use in-polymer flooding.

Embodiment 27. The polymer composition of any one of Embodiments 1 to 26, wherein the polymer composition includes one or more additional polymers, wherein, optionally, the one or more additional polymers comprise a tagging agent, and the fluorescence emission of the tagging agent may differ from the fluorescence emission of the first monomer of the copolymer.

Embodiment 28. The polymer composition of any one of Embodiments 1 to 27, wherein the polymer composition includes one or more additional ingredients, such as anionic surfactants (e.g. C₁₀₋₂₀ alkyl benzene sulfonates, C₁₀₋₂₀ olefin sulfonates, C₁₀₋₂₀ alkyl sulfates, C₁₀₋₂₀ alkyl 1 to 25 mole ether sulfates, C₁₀₋₂₀ paraffin sulfonates, C₁₀₋₂₀ soaps, C₁₀₋₂₀ alkyl phenol sulfates, sulfosuccinates, sulfosuccinamates, lignin sulfonates, fatty ester sulfonates, C₁₀₋₂₀ alkyl phenyl ether sulfates, C₁₀₋₂₀ alkyl ethanolamide sulfates, C₁₀-20 alpha sulfo fatty acid salts, C₁₀₋₂₀ acyl sarcosinates, isethionates, C₁₀₋₂₀ acyl taurides, C₁₀₋₂₀ alkyl hydrogen phosphates), non-ionic surfactants (e.g. ethoxylated and/or propoxylated C₁₀₋₂₀ alcohols, ethoxylated and/or propoxylated C₁₀₋₂₀ carboxylic acids, alkanolamides, amine oxides, and/or C₁₀₋₂₀ acyl sorbitan and/or glyceryl ethoxylates), amphoteric surfactants (e.g. betaines, sulfobetaines, and/or quaterised imidazolines), and/or cationic surfactants (e.g. benzalkonium salts, C₁₀₋₂₀ alkyl trimethyl ammonium salts, and/or C₁₀₋₂₀ alkyl trimethyl); sequestrants: chelating agents: corrosion inhibitors (e.g., imidazoline and quaterantry ammonium salts); and/or other threshold agents (e.g. polymers such as aminometholine phosphonate polymers, polyacrylic acid, or non polymeric agents such as sodium tripolyphosphate, sodium ethylenediamine tetracetate, sodium nitrilo triacetate, tetra potassium pyrophosphate, acetodiphosphonic acid and its salts, ammonium trismethylene phosphonic acid and its salts, ethylenediamine tetrakis(methylene phosphonic) acid and its salts, diethylenetriamine pentakis(methylene phosphonic) acid and its salts); tolyltriazole and mixtures of nitrate, benzqate, HHP and/or PTCB): hydrate inhibitors (e.g., methanol): cinetic inhibitors such as anti-agglomeration agents: biocides (e.g. tetrakis(hydroxymethyl) phosphonium salts, formaldehyde, glutaraldehyde, DENPA, bromopol isothiazoronal); oxidising biocides and/or bleaches (e.g. chlorine, chlorine dioxide, hydrogen peroxide, sodium perborate): foam controlling agents, such as silicone antifoams: oxygen scavengers such as hydrazines and/or hydroxylamines: pH controlling and/or buffering agents, such as amines, borates, citrates and/or acetates: chromium salts: zinc salts: asphaltene inhibitors; wax inhibitors: demulsifiers: other scale inhibitors; and/or other water treatment agents such as polymeric dispersants and coagulants including polymaleic, polyacrylic and polyvinylsulfonic acids and their salts, starches and/or carboxy methyl cellulose, and/or molybdates.

Embodiment 29. The polymer composition of any one of Embodiments 1 to 28, wherein the polymer composition includes two or more copolymers, wherein, optionally, each copolymer includes a different first monomer, and each of the different first monomers or molecules exhibits a different fluorescence emission.

Embodiment 30. The polymer composition of Embodiment 29, wherein the polymer composition includes (i) a first copolymer including a first monomer, which has a fluorescence emission maximum of about 500 to about 520 nm, (ii) a second copolymer including a first monomer, which has a fluorescence emission maximum of about 550 nm to about 590 nm, (iii) a third copolymer including a first monomer, which has a fluorescence emission maximum of about 640 nm to about 680 nm, or (iv) a combination thereof.

Embodiment 31. A method comprising providing a system comprising a fluid in circulation, wherein the fluid comprises the polymer composition of any one of Embodiments 1 to 30; measuring with an analytical technique an amount of the first monomer in the system or the fluid to determine an amount of the polymer composition in the system or the fluid, wherein the measuring is performed periodically or continuously; and optionally (i) adding an additional amount of the polymer composition to the system or the fluid if the amount of the polymer composition in the system or the fluid is less than a predetermined value, or (ii) removing a portion of the polymer composition from the system or the fluid if the amount of the polymer composition in the system or the fluid is greater than the predetermined value.

Embodiment 32. The method of Embodiment 31, wherein the method comprises (a) adding to a system or fluid a predetermined amount of a polymer composition as described herein: (b) periodically or continuously measuring the amount of tagging units (i.e., first monomer) in the system or fluid to determine an amount of the polymer composition in the system or fluid; and (c) periodically or continuously further adding more or removing a portion of the polymer composition to or from the system or fluid when the measured amount of tagging units (i.e., first monomer) is less than or greater than, respectively, a predetermined value.

Embodiment 33. The method of Embodiment 31 or 32, wherein the polymer composition is detected (e.g., measured) by fluorometry, such as with a fixed wavelength fluorometer.

Embodiment 34. The method of any one of Embodiments 31 to 33, wherein the amount of polymer added to the system is in the range of about 0.1 part per million to about 50,000 parts per million, about 0.1 part per million to about 25,000 parts per million, about 0.1 part per million to about 10,000 parts per million, about 0.1 part per million to about 1,000 parts per million, about 0.1 part per million to about 500 parts per million, or about 100 parts per million to about 200 parts per million, based on the capacity of the system.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

### Example 1—Tagged Composition for Polymer Flooding

In this example, fluorescein was copolymerized with acrylamide and sodium acrylate to form fluorescent tagged anionic polyacrylamide (APAM).

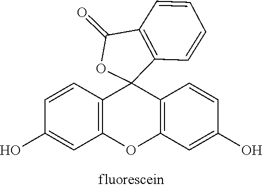 [SMILES: O=C1OC2(c3ccc(O)cc3Oc3cc(O)ccc32)c2ccccc21]

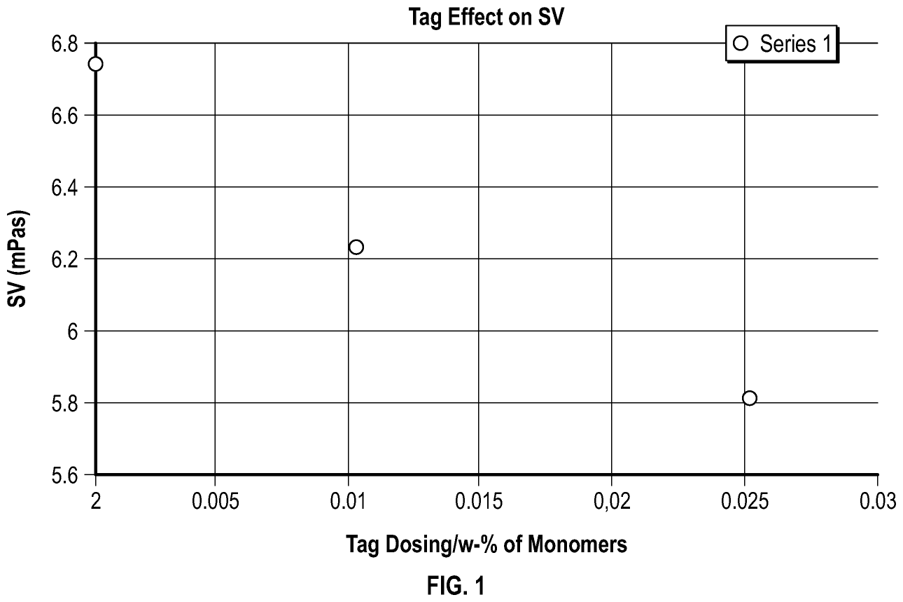

The resulting polymer exhibited a serum viscosity (SV) in a range similar to non-tagged polymers, as evidenced by the data of FIG. 1 and Table A.

TABLE A
Polymerization Results

| | Tag Dosing (Wt. % | | Insolubles (from |
|---|---|---|---|
| Sample | of Monomers) | SV (QC 20) | SV sieve) |
| 1 | 0.025 | 5.81 | 0 |
| 2 | 0.01 | 6.23 | 0 |
| 3 | 0 | 6.74 | 0.47 |

The analytical results confirmed that the tag of this example was polymerized, and was attached to the polymer backbone, likely as an end group. Not wishing to be bound by any particular theory, it is believed that the tag addition was achieved by a chain transition mechanism.

It was observed that SV decreased as the tag dosing increased (FIG. 1). The fluorescence yield of fluorescein was very high relatively, so a small tag dosing level could be used for polymer tagging.

The fluorescein tagged polymers of this example were prepared with the following procedure. Acrylamide, water, sodium acrylate, fluorescein, and additives were mixed together to form a monomer blend. The pH of the monomer blend was measured and adjusted to about 7.5.

The monomer blend was cooled down and degassed. A reducing agent (ferrous ammonium sulfate hexahydrate) was dissolved in acidified and degassed water. Concentrated sulphuric acid was used for the pH adjustment. An oxidizer (ammonium persulfate) was dissolved in degassed water. A complexing agent (DTPA-Na5) was dissolved in degassed water. A thermal initiator (V50) was dissolved in the degassed water. Redox initiators, a complexing agent, and thermal initiator were added to the degassed monomer blend to start polymerization. The polymerized gel was chopped and granulated into small pieces. The gel pieces were dried, ground, and sieved to obtain the final product.

The tagged polymers prepared by this example were fluorescent polymers (having an excitation max of about 490 to about 500 nm, an emission max of about 510 to about 520 nm, and was fluorescent active in neutral to alkaline pH).

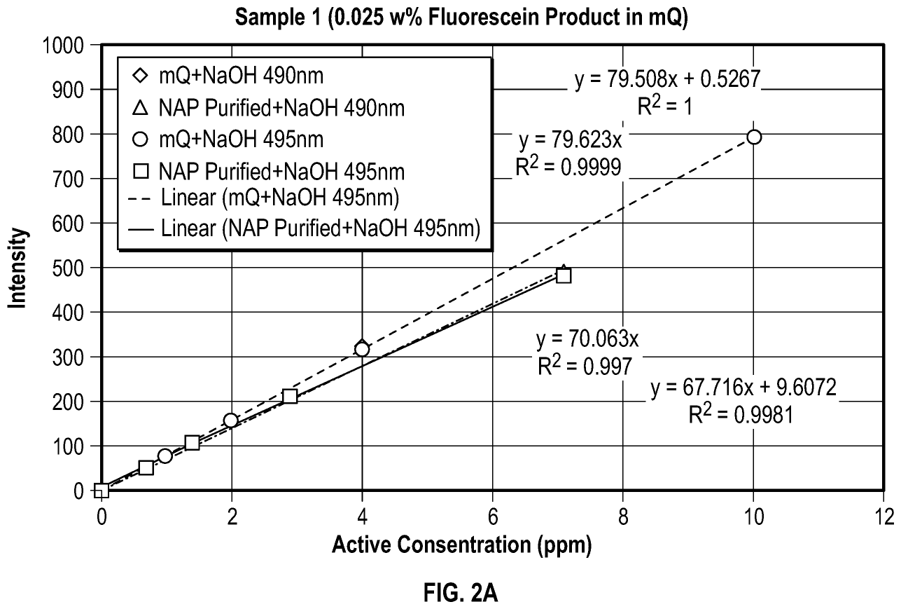

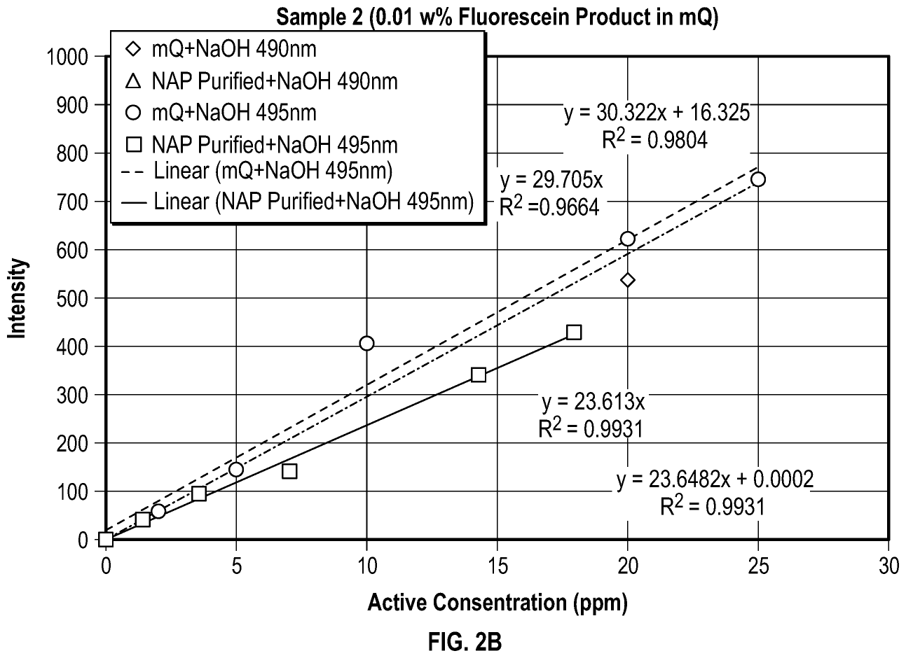

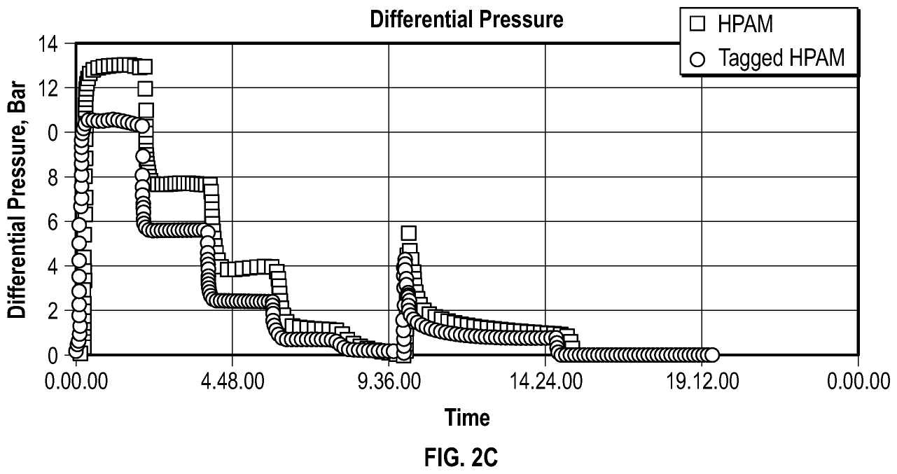

The fluorescence measurement confirmed that the polymers of this example had very good sensitivities. The attachment of the tag to the polymer backbone, as depicted at FIG. 2A and FIG. 2B, was estimated by comparing the fluorescence yield of clean and uncleaned samples. In FIG. 2A and FIG. 2B, excitation wavelengths are depicted in the legends. The samples were cleaned with a NAP™-25 size-exclusion chromatography (SEC) column (GE Healthcare, USA). The SEC column permitted polymeric materials to be separated from unattached tag monomers. As depicted at FIG. 2A and FIG. 2B, about 80 to about 88% (by weight) of the fluorescein tag was incorporated into the polymer backbone.

The results of this example confirmed that very high molecular weight polymer can be tagged.

### Example 2—Injectivity Test

The injectivity of a hydrolyzed tagged polymer of Example 1 (HPAM) and an untagged control hydrolyzed polymer were tested. The results, which are depicted at FIG. 2C, demonstrated that no plugging (i.e., no increase in differential pressure) was observed the untagged HPAM or the tagged HPAM.

1. A polymer composition comprising:
a copolymer comprising—
(i) a first monomer comprising (a) a compound of formula (I), formula (I′), or formula (I″), or (b) a compound or an isomer of formula (II), wherein the first monomer is a tagging monomer, and
(ii) at least one second monomer comprising at least one polymerizable double bond or at least one polymerizable triple bond; 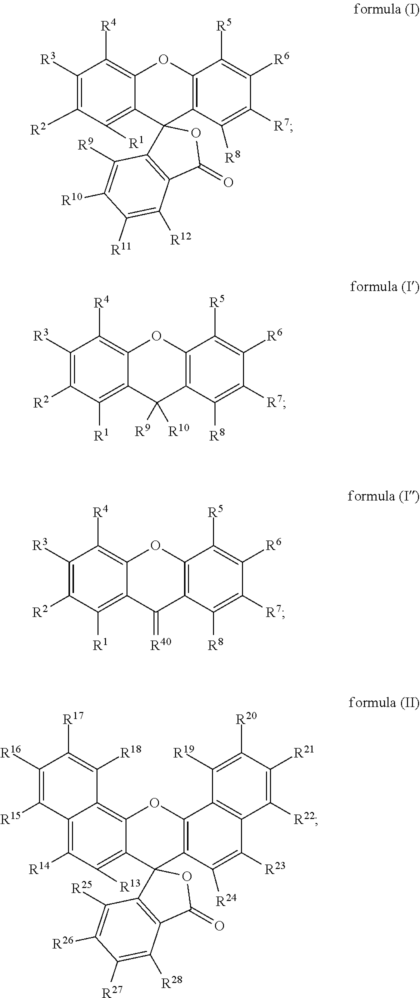
wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ are independently selected from the group consisting of hydrogen, hydroxyl, C₁-C₆ alkoxy, C₂-C₆ alkenoxy, C₂-C₆ alkynoxy, —N(R′)(R″), C₁-C₆ alkyl, C₂-C₆ alkenyl, C₂-C₆ alkynyl, and C₄-C₁₄ aryl,
wherein R⁴⁰ is selected from the group consisting of C₁-C₆ alkoxy, C₂-C₆ alkenoxy, C₂-C₆ alkynoxy, —N(R′), —N(R′)(R″), C₂-C₆ alkyl, C₂-C₆ alkenyl, C₂-C₆ alkynyl, and C₄-C₁₄ aryl,
wherein R′ and R″ are independently selected from the group consisting of hydrogen and C₁-C₆ alkyl, and
wherein the isomers of Formula (II) comprise a compound of Formula (IIi), a compound of Formula (IIii), a compound of Formula (IIiii), or a combination thereof—

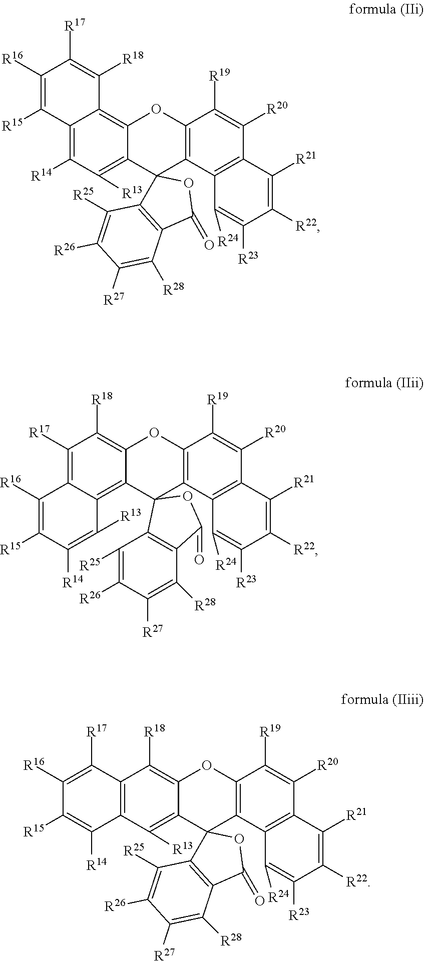

2. The polymer composition of claim 1, wherein the copolymer has a weight average molecular weight (Mw) of at least 1,000,000 Da.

3. The polymer composition of claim 1, wherein the first monomer is present in the copolymer at an amount of about 0.01% to about 5%, by weight, based on the weight of the copolymer.

4. The polymer composition of claim 1, wherein the at least one second monomer comprises acrylamide, sodium acrylate, or a combination thereof.

5. The polymer composition of claim 1, wherein the first monomer comprises the compound of formula (I), wherein R¹, R², R⁴, R⁵, R⁷, R⁸, R⁹, R¹⁰, R¹¹, and R¹² are hydrogen, or formula (I′) or (I″), wherein R¹, R², R⁴, R⁵, R⁷, and R⁸ are hydrogen.

6. The polymer composition of claim 5, wherein R³ and R⁶ are hydroxyl.

7. The polymer composition of claim 5, wherein R³ and R⁶ are —N(R′)(R″).

8. The polymer composition of claim 7, wherein R′ and R″ are independently selected from hydrogen and an unsubstituted C₁-C₆ alkyl.

9. The polymer composition of claim 1, wherein the first monomer comprises the compound or isomer of formula (II), wherein R¹³, R¹⁴, R¹⁵, R¹⁷, R¹⁸, R¹⁹, R²⁰, R²², R²³, R²⁴, R²⁵, R²⁶, R²⁷, and R²⁸ are hydrogen.

10. The polymer composition of claim 9, wherein R¹⁶ and R²¹ are hydroxyl.

11. The polymer composition of claim 9, wherein R¹⁶ and R²¹ are —N(R)(R″).

12. The polymer composition of claim 11, wherein R′ and R″ are independently selected from hydrogen and an unsubstituted C₁-C₆ alkyl.

13. The polymer composition of claim 1, wherein the copolymer is an anionic copolymer.

14. The polymer composition of claim 1, wherein the first monomer is an end group of the copolymer.

15. A method comprising:
providing a system comprising a fluid in circulation, wherein the fluid comprises the polymer composition of any one of claims 1 to 14;
measuring with an analytical technique an amount of the first monomer in the system or the fluid to determine an amount of the polymer composition in the system or the fluid, wherein the measuring is performed periodically or continuously; and
optionally (i) adding an additional amount of the polymer composition to the system or the fluid if the amount of the polymer composition in the system or the fluid is less than a predetermined value, or (ii) removing a portion of the polymer composition from the system or the fluid if the amount of the polymer composition in the system or the fluid is greater than the predetermined value.
